(19) United States
(12) Patent Application Publication
(10) Pub. No.: US 2011/0251138 A1
(43) Pub. Date: Oct. 13, 2011

(54) LIPOIC ACID METABOLITE CONJUGATE: PREPARATION AND THEIR THERAPEUTIC EFFECT

(75) Inventor: Ravikumar Kabyadi Seetharama, Redwood City, CA (US)

(73) Assignee: Ravikuma Kabyadi Seetharama, Redwood City, CA (US)

(21) Appl. No.: 12/796,099

(22) Filed: Jun. 8, 2010

Related U.S. Application Data
(60) Provisional application No. 61/341,975, filed on Apr. 8, 2010.
(60) Provisional application No. 61/341,977, filed on Apr. 8, 2010.
(60) Provisional application No. 61/343,069, filed on Apr. 23, 2010.

(51) Int. Cl. A61K 31/385; C07D 339/04; C07K 7/64; C07D 475/08; A61K 31/4436; A61K 38/12; A61K 31/519; A61P 35/00; A61P 3/10; A61P 25/00; A61P 1/16; A61P 43/00; C12N 5/02; G01N 1/30; C07D 409/02

(52) U.S. Cl. 514/21.1; 546/280.7; 549/39; 530/321; 544/260; 514/336; 514/440; 514/249; 435/375; 435/29; 435/40.5

(57) ABSTRACT

The present invention is a design and synthesis of a series of therapeutic conjugates which consists of tautomers of lipoic acid metabolites, with small molecule, vitamin, carbohydrates, peptides, chemotherapeutic agent wherein or not the conjugate possesses dual binding ability. The present invention can be used to therapeutics and diagnostics in vitro for cancer and other diseases associated with altered metabolic enzymes. The invention can also be used for the controlled release of more stable form of lipoic acid in its salt form with the minerals or vitamins.

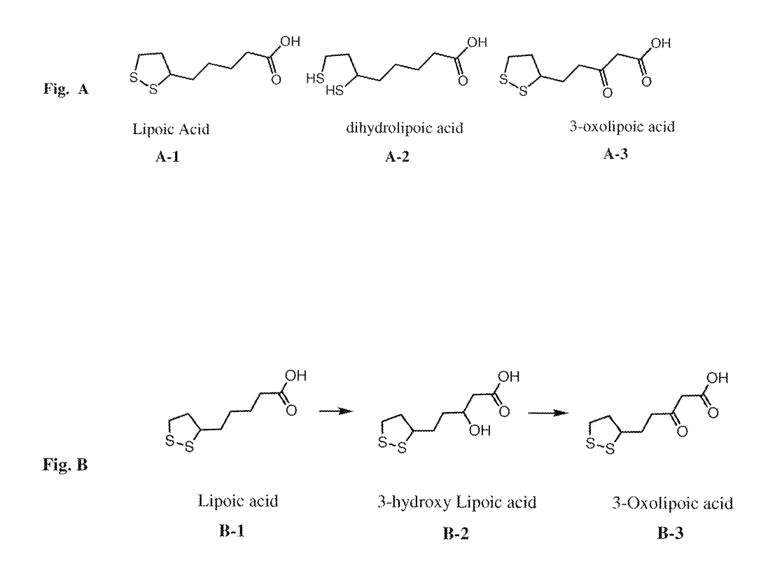

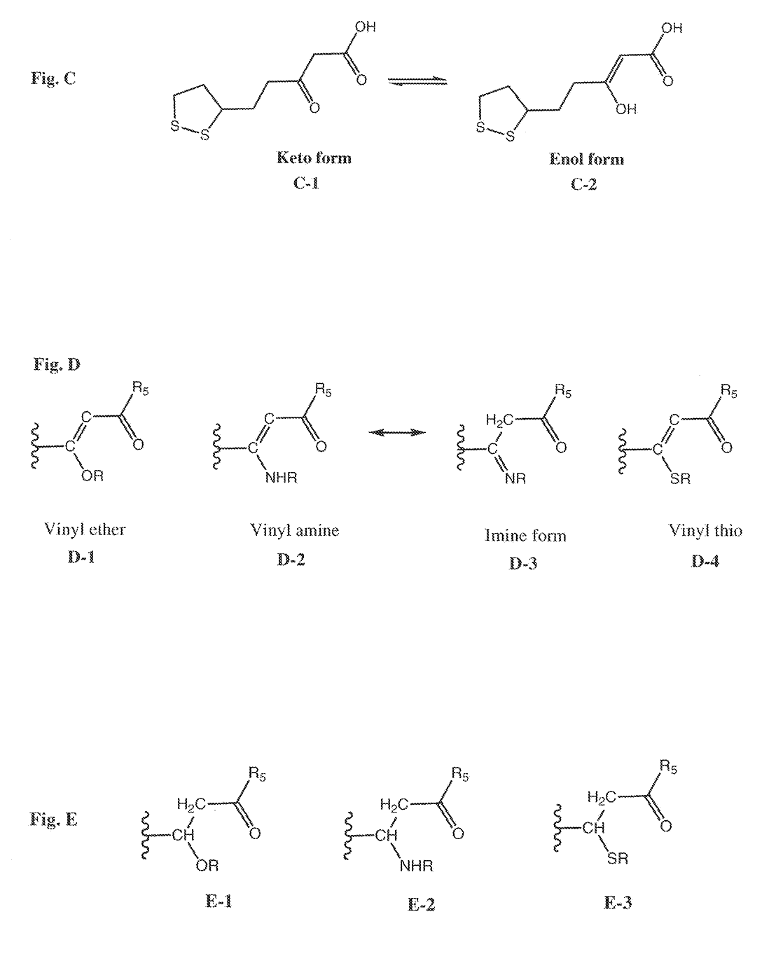

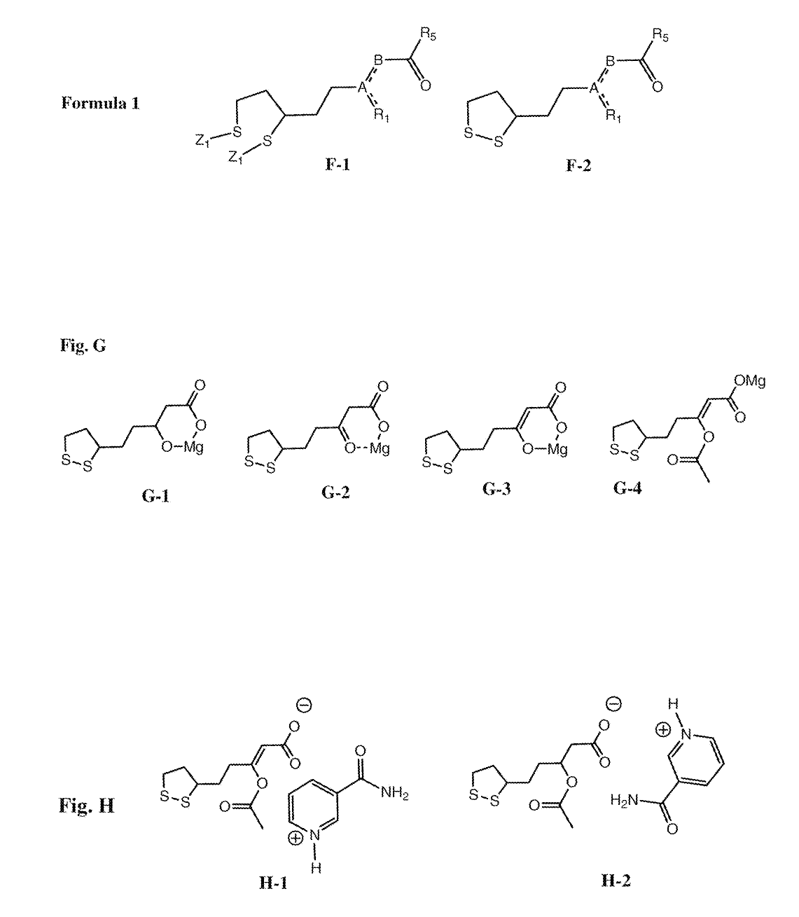

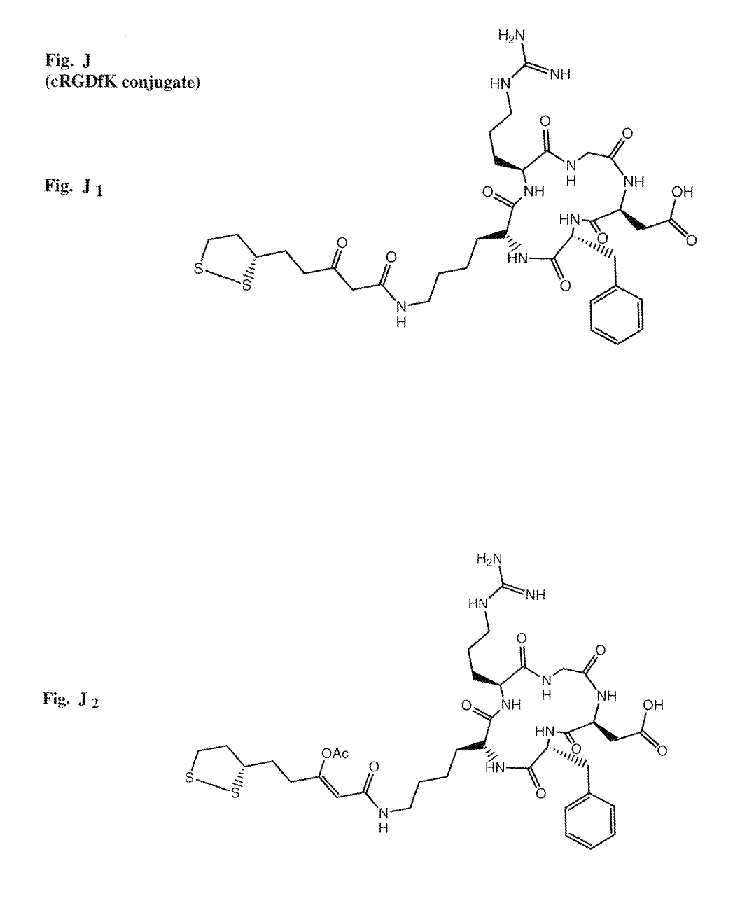

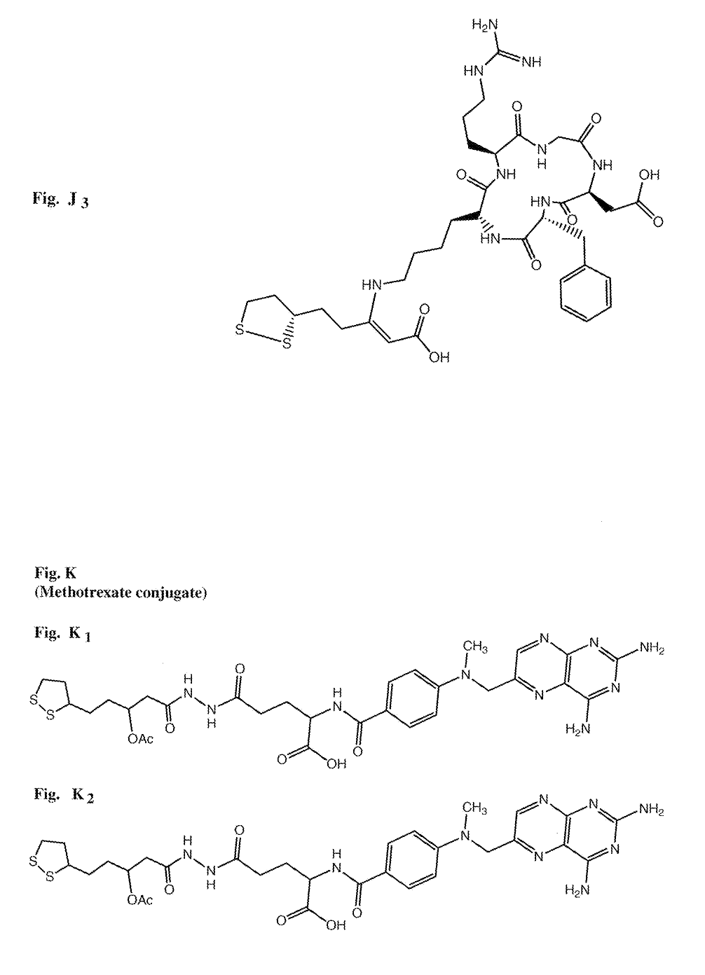

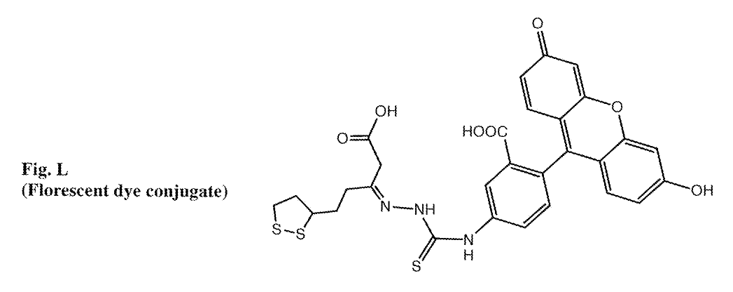

This application claims priority to provisional patent application U.S. Ser. No. 60/923,774 filed Apr. 17, 2007; 60/923,775 filed Apr. 17, 2007; 61/341,975 filed Apr. 8, 2010; 61/341,977 filed Apr. 8, 2010; 61/343,069 filed Apr. 23, 2010.

### FIELD OF THE INVENTION

The present invention is a design and synthesis of a series of therapeutic conjugates which consists of lipoic acid metabolites, 3-oxolipoic acid and 3-hydroxy lipoic acid and its conjugate with small molecule, vitamin, carbohydrates, peptides, chemotherapeutic agent wherein or not the conjugate possesses dual binding ability.

The present invention relates to therapeutics and diagnostics in vitro for cancer and other diseases associated with altered metabolic enzymes. In particular, the invention relates to a novel class of therapeutic agents which selectively target and kill tumor cells, and certain other types of cells involved in diseases processes.

The targeted delivery can be used, for example, for disease sensing, imaging, and drug delivery. The present invention also relates to a method and a kit to prepare, as well as a method to use, such conjugates.

This invention relates to the coenzyme lipoic acid metabolite and more particularly to a method of stabilizing R-Lipoic acid metabolite with magnesium

### BACKGROUND OF THE INVENTION

Glutathione is found throughout the tissues of the living body, is a major reducing agent in cells, and plays a very important role in the oxidation-reduction metabolic processes. In particular, the reduced glutathione (GSH) plays a key role in various cellular defense and repair mechanisms. GSH reduces peroxide and the oxidized form of glutathione, GSSG will be reduced by NADPH to regenerate GSH.

α-lipoic acid is an antioxidant, and is widely used in prevention of various diseases. Its main function is to increase production of glutathione, which helps dissolve toxic substances in the liver. An antioxidant helps neutralize free radicals in vivo and protects the cells from damage. A free radical is an unstable molecule that tries to steal electrons from other molecules, thus causing damage. Smoking, pollution, car exhaust, fried foods are some sources of free radicals. Alpha lipoic acid can be found in foods such as meats, and veggies (i.e. Spinach). Alpha lipoic acid is also easily absorbed into the blood stream.

LA is one of the most important molecules in redox signaling due to maintenance of oxidizing conditions by stabilizing disulfides in the extra cellular surface while the intracellular environment is maintained in the reduced state with the help of free sulfhydryl groups.

Alpha-lipoic acid is a chiral molecule and is known by a variety of names including thioctic acid; 1,2-diethylene-3-pentanoic acid; 1,2-diethylene-3-valeric acid; and 6,8-thiooctic acid. Alpha-lipoic acid was tentatively classified as a vitamin after its isolation, but it was later found to be synthesized by animals and humans. The complete enzyme pathway that is responsible for the de novo synthesis has not yet been definitively elucidated.

In US Patent Application Publication No. 2001/0039292 A1, US 2002/0107234 A1 and US 2004/0092586 A1 studied about various lipoic acid analogs and derivatives. In U.S. Pat. No. 5,650,429 and EP 0855396 A1 discussed about oxidized and reduced form of metabolite of lipoic acid.

There is evidence that the two optical isomers of alpha-lipoic acid have different biological activities. R-alpha-lipoic acid occurs naturally in plants and animals and is the only form that functions as a cofactor for mitochondrial enzymes. Chemical synthesis of alpha-lipoic acid results in a 50/50 or racemic mixture of S-alpha-lipoic acid and R-alpha-lipoic acid. Within the mitochondria, R-alpha-lipoic acid is reduced to DHLA, the more potent antioxidant, 28 times faster than S-alpha-lipoic acid. However, in the cytosol S-alpha-lipoic acid is reduced to DHLA twice as fast as R-alpha-lipoic acid. One study in humans found R-alpha-lipoic acid to be more bioavailable than S-alpha-lipoic acid when taken orally. R-alpha lipoic acid was more effective than S-alpha-lipoic acid in enhancing insulin-stimulated glucose transport and metabolism in insulin-resistant rat skeletal muscle, and R-alpha-lipoic acid was more effective than racemic alpha-lipoic acid and S-alpha-lipoic acid in preventing cataracts in rats.

Various enantiomeric forms of alpha-lipoic acid and combinations and derivatives thereof (including its reduced form), have been used to treat numerous conditions. For example, U.S. Pat. Nos. 5,650,429 and 5,532,269 disclose the use of lipoic acids in the treatment of circulatory disorders. U.S. Pat. No. 5,569,670 discloses combinations of lipoic acids and vitamins in compositions useful for producing analgesic, anti-inflammatory, antinecrotic, anti-diabetic and other therapeutic effects. U.S. Pat. No. 5,334,612 describes certain alkylated derivatives of lipoic acid and their use in treatment of retroviral diseases. U.S. Pat. No. 5,084,481 discloses the use of reduced lipoic acid (dihydrolipoic acid, DHLA) and salts thereof in treating inflammatory diseases. U.S. Pat. No. 6,693,664 discloses use of lipoic acid and dihydrolipoic acid in the treatment of diabetes. U.S. Pat. No. 5,508,275 discloses a variety of lipid-selective antioxidants, including lipoic acid derivatives.

Lipoic acid readily crosses the blood-brain barrier and accumulates in all neuronal cell types. (Packer, L.; Tritschler, H. J.; Wessel, K. Free Radic. Biol. Med. 22, 359-378 (1997). There, cytosolic and mitochondrial dehydrogenases rapidly reduce it to dihydrolipoic acid (DHLA).

More recently, Goralska and Co-workers reported beneficial effects of lipoic acid treatment in preventing iron accumulation in lens epithelial cells. (Goralska, M; Dackor, R.; Holley, B.; McGahan, M. C. Exp. Eye Res. 76, 241-248 (2003) Thus, it is plausible that lipoic acid may also be beneficial in normalizing the adverse effects or iron accumulation in the aging brain.

In the past, researchers are exploring lipoic acid and dihydrolipoic acid analogs and derivatives for various applications. However, our intention is to explore the use of oxidized lipoic acid metabolites (3-oxo lipoic acid and 3-hydroxy lipoic acid) and its applications.

Cancer can be defined as a disease that is characterized by a failure of the control mechanisms that are involved in cell division. It is very well known that the cancer cells have great appetite for food, particularly carbohydrate, vitamin and minerals. The development of new blood vessels, Angiogenesis, plays a central role in the pathogenesis of cancer. It is crucial for maintaining the supply of oxygen and nutrients to support tumor growth. Rapidly proliferating cells have a higher demand for DNA and consequently require large quantities of purine, pyrimidine, amino acids and carbohydrates substrates.

For anti-tumor therapy different strategies have been employed, e.g., radiotherapy, chemotherapy, or immunotherapy. Notably, these approaches do not only address the tumor cells themselves, but also the tumor stroma cells, e.g., endothelial cells, fibroblasts, and macrophages. This is of advantage, since these cells actively contribute to the proliferative and invasive behavior of the tumor cells via secretion of growth factors, angiogenic factors, cytokines, and proteolytic enzymes. In addition, tumor stroma cells take part in immune evasion mechanisms of cancer. Thus, approaches targeting the tumor.

Stroma attracts increasing attention as anti-cancer therapy. Several molecules including growth factors (e.g., VEGF, CTGF), growth factor receptors (CD105, VEGFRs), adhesion molecules (avb3 integrin), and enzymes (CAIX, FAPa, MMPs, PSMA, uPA) are all expressed in tumor cell. Consequently, these molecules can be targeted by inhibitors as well as by active and passive immunotherapy to treat cancer. (Medicine in development of Cancer by Billy Tauzin, PhRIVIA, 2009, 1-104).

Many human cancer cell lines have been found to have highly over-expressed levels of the protein which binds folic acid. (Weitman, S. D., Lark, R. H., Coney, L. R., Fort, D. W., Frasca, V. Zurawski, Jr., V. R., and Kamen, B. A. Cancer Research 52, 3396-3401 (1992); Ross, J. F., Chaudhuri, P. K., and Ratnam, M. Cancer 73, 2432-2443 (1993); Prasad, P. D., Ramamoorthy, S., Moe, A. J., smith, C. H., Leibach, F. H., and Ganaphthy, V. Biochim. Biophys. Acta 1223, 71-75 (1994); Li, P. Y, Vecchio, S. D., Fonti, R., carriero, M. V., Potena, M. I., Botti, G., Miotti, S., Lastoria, S., Menard, S., Colnaghi, M. I., and Salvatore, M. J. Nuclear Med. 37, 665-672 (1996); Antony, A. C. Annu Rev. Nutr. 16, 501-521 (1996); Bueno, R., Appasani, K., Mercer, H., Lester, S., and Sugarbaker, D. J. Thoracic Cardio. Sur. 121, 225-233 (2001)). These facts have attracted considerable attention (Reddy, J. A. and Low, P. S. Critical Reviews in Therapeutic Drug Carrier Systems 15, 587-627 (1998); Drummond, D. C., Hong, K., Park, J. W., Benz, C. C., and Kirpotin, D. B. Vitamins and Hormones 60, 285-332 (2001); Sudimack, J. B. A. and Lee, R. J. Advanced Drug Delivery Reviews 41, 147-162 (2000)) and have been exploited for developing cancer selective drug delivery system (DDS) (Lee, R. J. and Low, P. S. J. Biol. Chem. 269, 3198-3204 (1994); Lee, R. J. and Low, P. S. Biochim. Biophys. Acta 1233, 134-144 (1995); Rui, Y., Wang, S., Low, P. S., and Thompson, D. H. J. Am. Chem. Soc. 120, 11213-11218 (1998); Goren, D., Horowits, A. T., Tzemach, D., Tarshish, M., Zalipsky, S., and Gabizon, A. Clinical Cancer Research 6, 1949-1957 (2000)).

A number of researchers have compared the number of folate-receptors on malignant tissue versus normal tissue. (Mantovani, L. T.; Miotti, S.; Menard. S.; Canevari, S.; Raspagliesi, F.; Bottini, C.; Bottero, F.; Colnaghi, M. I.; Eur. J. Cancer 30A, 363-369 (1994); Ross, J. F.; Chaudhuri, P. K.; Ratnam, M. Cancer, 73, 2432-2443 (1994). It shows that malignant ovarian, endometrial, and brain tissues all have significantly higher numbers of folate receptors than normal tissues. Folate receptors have also been detected in breast, lung, colon, kidney, and head/neck cancers.

One of the present inventions is a method of stabilizing Magnesium salt of lipoic acid analog. Magnesium is a natural mineral for which supplementation often is necessary or desirable. Magnesium is a very important mineral in the body. Magnesium plays a vital role in enzymatic reactions and in maintaining the intracellular ionic milieu. Without adequate intracellular magnesium, muscles are weak, the heartbeat irregular and brain function affected.

Magnesium is needed for more than 300 biochemical reactions in the body. It helps maintain normal muscle and nerve function, keeps heart rhythm steady, and bones strong. It is also involved in energy metabolism and protein synthesis.

While magnesium deficiency is fairly common, it is frequently overlooked as a source of problems. The reason is that serum magnesium levels (the test most doctors use) do not reflect actual body stores of magnesium. Blood levels are kept within the normal range at the expense of other tissues. When deficiency does occur, it is usually due to excessive loss of magnesium in urine, gastrointestinal system disorders that cause a loss of magnesium or limit magnesium absorption, or a chronically low intake of magnesium.

Treatment with diuretics (water pills), some antibiotics, and some cancer medicines such as Cisplatin, can increase the loss of magnesium in urine. Poorly controlled diabetes increases loss of magnesium in urine, causing a depletion of magnesium stores.

Gastrointestinal problems, such as malabsorption disorders, can cause magnesium depletion by preventing the body from using the magnesium in food. Chronic or excessive vomiting and diarrhea may also result in magnesium depletion. The loss of magnesium through diarrhea and fat malabsorption usually occurs after intestinal surgery or infection, but it can occur with chronic malabsorptive problems such as Crohn's disease, gluten-sensitive enteropathy, and regional enteritis. Individuals with these conditions may need extra magnesium.

Signs of magnesium deficiency include confusion, disorientation, loss of appetite, depression, muscle contractions and cramps, tingling, numbness, abnormal heart rhythms, coronary spasm and seizures.

Diets may be deficient in magnesium for a variety of reasons. The amount of magnesium in the soil in which the food is grown may be reduced. Magnesium can be lost in the processing and refining of foods and in making oils from the magnesium-rich nuts and seeds. Nearly 85% of the magnesium in grains is lost during the milling of flours. Soaking and boiling foods can leach magnesium into the water, so the “pot liquor” from cooking vegetables may be high in magnesium and other minerals. Oxalic acid in vegetables such as rhubarb, spinach and chard, and phytic acid in some grains may form insoluble salts with magnesium, causing it to be eliminated rather than absorbed. Supplemental Vitamin D or Calcium reduces magnesium uptake. Diets high in phosphorus (meat, milk products, lentils, seeds, beans, nuts, chocolate, peanut butter, bran) reduce magnesium uptake. Magnesium elimination is increased in people who use alcohol, caffeine or excess sugar, or who take diuretics or birth control pills. Magnesium is absorbed toward the end of the small intestine. If any portion of this area (called the ileum) has been removed, magnesium absorption will be reduced.

Lipoic acid suffers from certain disadvantages; however, in particular, the natural form R-lipoic acid is unstable above 40° C., so it can degrade under some warehousing conditions. Also Lipoic acid is hygroscopic, absorbing moisture and subsequently degrading. What is needed is stabilization of this natural form of lipoic acid in a natural salt capable of maintaining potency at higher temperature.

For the avoidance of doubt, the compounds of lipoic acid and its metabolite has the formula as shown in FIG. A.

It is believed that lipoic acid initially oxidized in vivo to 3-hydroxy lipoic acid which further oxidized to 3-oxo lipoic acid as shown in FIG. B.

The present inventions identify the keto-enol tautomers of the metabolite of lipoic acid as shown in FIG. C.

The present invention uses the ene-derivative of the lipoic metabolite as shown in FIG. D.

The present invention use the reduced form of ene derivative of the lipoic acid as shown in FIG. E

The compounds of the present invention are those compounds of Formula 1

### SUMMARY OF THE INVENTION

It is an object of the present invention to provide a more stable formulation of lipoic acid and to make lipoic acid readily usable by the body.

In one aspect of this invention is the availability of magnesium source in the form of magnesium salt of lipoic metabolite as shown in FIG. G.

Another aspect of this invention is to make the salt with natural amines. For example, Niacinamide is considered as Vitamin B₃ and the metabolite of lipoic acid forms salt with such vitamin.

Yet another aspect of this invention is the use of lipoic acid metabolite for drug transport.

Other aspect of this invention is the use of lipoic acid-dye derivative for imaging in vitro

One preferred class of compounds comprise the structure of Formula 1:

wherein:

A and B are carbon atoms directly connected to carbon-carbon single bond, carbon-carbon double bond

Z₁ is H, COCH₃;

R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄;

R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙCₘH₂ₘ₊₁; —(CH₂CH₂CH₂O)ₙCₘH₂ₘ₊₁; —X(CH₂—CH₂O)ₙCₘH₂ₘ₊₁Y; X(CH₂CH₂CH₂O)ₙCₘH₂ₘ₊₁Y; n is 0-16; m is 0-16; X is CO, COO, CH₂O, CONHNH; Y is OH, NH₂, SH, COOH, SO₃H, H₂PO₄

R₃ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH

R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol.

R₅ is OH; OLi, ONa; OK, OMg, OMn, OCu, OCa, OAl, OFe, OAg, OAu, O-Niacinamide salt, O-Thiamine salt; OZR₆, ZR₇R₈, ZR₈, NR₉SO₂R₁₀ and Z is O, S, NH, NHNH.

R₆ denotes -alkyl CₙH₂ₙ₊₁; -alkene CₘH₂ₘ; -alkyne CₘH₂ₘ₋₂; —(CH₂CH₂O)ₙCₘH₂ₘY; —(CH₂CH₂CH₂O)ₙCₘH₂ₘY; n is 0-16; m is 0-16; Y is H, OH, NH₂, SH, COOH

R₇ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH

R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol

R9 denotes R₆, R₇ R₈, R₈

R₁₀ denotes R₆, R₇R₈, R₈

### BRIEF DESCRIPTION OF THE DRAWINGS

FIG. A shows the structure of lipoic acid and its metabolites.

FIG. B shows the oxidation of lipoic acid.

FIG. C shows the keto-enol tautomers of the metabolites of FIG. B.

FIG. D shows the ene-derivatives of the lipoic metabolites.

FIG. E shows the reduced form of the ene-derivatives.

FIG. F shows the formula of compounds of the present invention.

FIG. G shows magnesium salts of lipoic metabolites.

FIG. H shows niacinamide derivatives of lipoic acid.

FIG. J shows cRGDfk conjugates.

FIG. K shows methotrexate conjugates.

FIG. L shows florescent dye conjugates.

### DETAILED DESCRIPTION OF THE PREFERRED EMBODIMENTS

### Structural Characteristics of Conjugate

One aspect of the present invention is 3-oxo lipoic acid and 3-hydroxy lipoic acid conjugates of the Formula 1 are a carrier for fluorescent dye molecule for the application in directed toward a method of imaging in vitro.

Another aspect of the present invention is 3-oxo lipoic acid and 3-hydroxy lipoic acid conjugates for the Formula 1 are a carrier for chemotherapeutic drug for effective delivery.

The method of imaging may include fluorescence imaging, radiologic imaging or the targeted delivery of radioisotopes. The targeted delivery of radioisotopes may include targeted delivery of radioisotopes to tissues such as tumors.

The alkyl group preferably comprises CₙH₂ₙ₊₁ wherein n is 0-16. Such alkyl groups may be substituted with the moieties such as for example OH, NH₂, and Cl. Examples of alkyl groups include but are not limited to methyl, ethyl, butyl, decanyl.

An alkene may preferably comprise CₙH₂ₙ wherein n=2-16. Examples of alkene groups include but are not limited to propylene, cyclopropene.

The alkyne may preferably comprise CₙH₂ₙ₋₂ wherein n is 2-16. Examples of alkyne groups include but are not limited to propyne, acetylene.

Alkyl, alkene and alkyne groups can have additions on any of their carbons. Examples of additions include but are not limited to hydroxyls and amines.

### DEFINITIONS

The term “lipoic acid’ is intended to mean α-lipoic acid which is a chiral molecule also known as thioctic acid; 1,2-diethylene-3 pentanoic acid; 1,2-diethylene-3 valeric acid; and 6,8-thioctic acid. Unless specified the term covers the racemic mixture as well as any other (non-50-50) mixture of the enantiomers including substantially pure forms of either the R-(+) or the S-(−) enantiomer. Further, unless specified otherwise the term covers pharmaceutically acceptable salts (e.g. Na and K salts) and amides, esters and metabolites of the acid.

The terms “treating”, and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting it's development; or (c) relieving the disease, i.e. causing regression of the disease and/or it's symptoms or conditions. The invention is directed towards treating patient's suffering from disease related to abnormal tissues. The present invention is involved in preventing, inhibiting, or relieving adverse effects attributed to cancer tissues

The term “excipient material” is intended to mean any compound forming a part of the formulation which is intended to act merely as a carrier i.e. not intended to have biological activity itself.

The term “quick release formulation” refers to a conventional oral dosage formulation. Such a formulation may be a tablet, capsule or the like designed to provide for substantially immediate release of the active ingredient and includes enteric coated oral formulation which provides some initial protection to the active ingredient and thereafter allow substantially immediate release of the entire active ingredient. A quick release formulation is not formulated in a manner so as to obtain a gradual, slow, or controlled release of the active ingredient.

Methods for Using Formula 1 Compounds

The material of the present invention may be used in a method for preventing or inhibiting diseases involving altered or distinct cellular activity. Such diseases are characterized by sensitivity to the lipoate compositions of the present invention. Cells with appropriately altered are particularly targeted and killed, while surrounding healthy tissues remain unharmed by the material.

In a preferred treatment method, the instant material compositions may be useful for the preventing and treatment of cancers such as primary or metastatic melanoma, thymoma, lymphoma, Hodgkin's lymphoma, leukemias, uterine cancer, cervical cancer, bladder cancer, kidney cancer, colon cancer, and adenocarcinomas such as breast cancer, prostate cancer, ovarian cancer, pancreatic cancer.

The preferred dosage of the material, or pharmaceutical composition thereof, is selected based on other criteria, including the particular composition employed and the age, weight, and condition of the individual. Importantly, the quantity of Formula 1 compounds used should be sufficient to inhibit or kill tumor cells while leaving normal cells substantially unharmed.

By adapting the treatments described herein, the Formula 1 compound may also be used in methods for treating diseases other than cancer, where the disease-causing cells exhibit altered metabolic patterns. For example, eukaryotic pathogens of humans and other animals are generally much more difficult to treat than bacterial pathogens because eukaryotic cells are so much more similar to animal cells than are bacterial cells.

Another important application of this Formula 1 compounds may be used as diagnostic agents in vitro. As stated earlier, depending on the specific tumor cell or cell type in question, different conjugates may be more or less effective at inhibiting distinct tumor classes.

Compositions of Formula 1 for Therapeutic Use

In the methods of preventing or inhibiting cancer, the Formula 1 compounds or a pharmaceutical composition comprising a Formula 1 compounds, may be administered via one of several routes including intravenous, intramuscular, subcutaneous, intradermally, intraperitoneal, intrathoracic, intrapleural, intrauterine, topical, or intratumor.

Those skilled in the art will recognize that the mode of administering the Formula 1 compounds depends on the type of cancer, or symptom to be treated. For example, a preferred mode of administering the Formula 1 compounds for treatment of leukemia would involve intravenous administration, whereas preferred methods for treating skin cancer would involve, for example, topical or intradermal administration.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Before the present, formulations, methods and components used therein are disclosed and described, it is to be understood that this invention is not limited to particular compounds, excipients or formulations as such may of course, vary. It is also to be understood that the terminology used herein is for the purpose of describing particular embodiments only, and is not intended to be limiting, since the scope of the present invention will be limited only by the appended claims.

For therapeutic applications, a pharmaceutical composition may be in the form of tablets, capsules, powders, granules, suppositories, reconstitutable powders, or liquid preparations such as oral or sterile parenteral solutions or suspensions. For oral administration, tablets and capsules may contain conventional excipients, such as binding agents, tabletting lubricants, pharmaceutically acceptable wetting agents such as sodium lauryl sulphate.

For parenteral administration, fluid unit dosage forms may be prepared utilizing the Formula 1 compounds and a sterile vehicle, and, depending on the concentration used, can either be suspended or dissolved in the vehicle. In preparing solutions, the Formula 1 compounds can be dissolved in water for injection and filter sterilized before filling into a suitable vial or ampoule and sealing. Also, adjuvant such as local anesthetic, a preservative, and buffering agents can be dissolved in the vehicle. To enhance stability, the composition can be frozen after filling into the vial and the water removed under vacuum. A surfactant or wetting agent can be included in the composition to facilitate uniform distribution of the Formula 1 compounds.

### Experimental Procedure

General Procedure for the Synthesis of Lipoic Metabolite-Natural Amine Salt

Method 1: Synthesis of compound H-1 (2-ene-3-O-acetyl lipoic acid-Niacinamide Salt): 1.4 g of Niacinamide was added into a solution of 2.6 g of 2-ene-3-O-acetyl lipoic acid in Toluene (30 mL) and the resultant solution was warmed to make homogeneous solution and stirred for 30 minutes. The resultant solution was cooled to room temperature and the yellow crystals obtained were filtered, dried under vacuum to afford 3.1 g of the desired salt.

Method 2: Synthesis of compound H-2 (3-O-acetyl lipoic acid—Thiamine salt): A solution of 2.64 g of 3-O-acetyl lipoic acid in methanol (30 mL) was added into the stirred solution of 1.4 g of Niacinamide in methanol (30 mL) and the resultant mixture was stirred for 30 minutes. The resultant solution was concentrated under vacuum and triturated with tert-butyl methyl ether, filtered and dried under vacuum to afford 2.6 g of pale yellow crystalline powder.

General Procedure for the Synthesis of Lipoic Metabolite-Magnesium Salt

Synthesis of Compound G-4 (2-ene-3-O-acetyl lipoic—Magnesium salt): 1.14 g of magnesium ethoxide was added into a solution of 2.6 g of 2-ene-3-O-acetyl lipoic acid in ethanol (50 mL) the resulting mixture was stirred for 10 minutes, concentrated under vacuum, cooled and filtered to obtain 2.1 g of the product as crystalline solid.

General Procedure for the Direct Coupling of 3-Hydroxylipoic Acid with Amine Reactive Ligand:

Method 1: 3-hydroxylipoic acid (1 equivalent) and amine reactive ligand (1.2 eq) were taken together in tetrahydrofuran and into this solution N,N′-diisopropyl carbodimide (1.2 eq) and diisopropyl ethyl amine (1.2 eq) were added and the reaction mixture was stirred for 5 to 12 hrs. The reaction mixture was quenched with water and stirred for 3 to 5 hrs. Filtered the solution and the filtrate was extracted with ethyl acetate and the organic layer was isolated and washed with water, dried over anhydrous magnesium sulfate and filtered. The filtrate was evaporated to dryness and the crude product was purified through silica gel column chromatography to afford the pure product.

Method 2: 3-hydroxylipoic acid (1 equivalent) and HATU were taken together in acetonitrile and into this solution amine reactive ligand (1.2 equivalents) and N-methyl morpholine (2 equivalents) were added and stirred for 6 to 16 hrs. The reaction mixture was quenched with water and the resultant mixture was extracted with ethyl acetate. The organic layer was isolated, dried over magnesium sulfate, filtered and evaporated to dryness to afford the desired conjugate.

Synthesis of 3-oxolipoic-NHS ester: To a stirred solution of 3-oxolipoic acid (11 g, 50 mmol) in dry acetonitrile (100 mL) were added N,N′-disuccinimidyl carbonate (19.2 g, 75 mmol) and triethylamine (21 mL, 150 mmol). The resulting mixture was stirred at 25° C. for 2 hr. The mixture was concentrated under reduced pressure and the residue was diluted with aqueous NaHCO₃ solution (200 mL) and extracted thoroughly with Ethyl acetate (3×75 mL). The combined extracts were washed with brine and dried over anhydrous MgSO₄ and filtered. Evaporation of the solvent provided the activated ester as a pale yellow solid (11.9 g, 75%)

Synthesis of 3-oxolipoic acid-cRGDfK conjugate: 3-oxolipoic acid (1.1 g, 5 mmol) and O-(7-Azabenzotriazol-1-yl)-N,N,N′,N′-tetramethyluronium hexafluoro phosphate, HATU (2.28 g, 6 mmol) were taken together in acetonitrile (25 mL) and into this solution a mixture of cRGDfK.2TFA (4.15 g, 5 mmol) and N-methyl morpholine (1.01 g, 10 mmol) was added and stirred for 16 hrs. The reaction mixture was quenched with water and the reaction mixture was extracted with ethyl acetate (3×50 mL). The combined organic layer was dried over anhydrous magnesium sulfate, filtered and evaporated to dryness. The crude product obtained was purified through silica gel column chromatography and the pure product containing fractions were combined and evaporated to dryness to afford pale yellow solid (3.3 g, 82%)

Synthesis of 3-hydroxylipoic NHS ester: To a stirred solution of 3-hydroxylipoic acid (2.22 g, 10 mmol) in dry acetonitrile (50 mL) were added N,N′-disuccinimidyl carbonate (3.1 g, 12 mmol) and triethylamine (4.2 mL, 30 mmol). The resulting mixture was stirred at 25° C. for 2 hr. The mixture was concentrated under reduced pressure and the residue was diluted with aqueous NaHCO₃ solution (100 mL) and extracted thoroughly with Ethyl acetate (3×60 mL). The combined extracts were washed with brine and dried over anhydrous MgSO₄ and filtered. Evaporation of the solvent provided the activated ester as a pale yellow solid (2.8 g, 83%)

Synthesis of 3-hydroxylipoic hydrazide: Hydrazine hydrate (2.5 mL) in MeOH (25 mL) was added to a solution of 3-hydroxylipoic NHS ester (1.6 g, 5 mmol) in MeOH (20 mL), and the solution was stirred at room temperature for 4 hrs. After vacuum evaporation of most of the solvent, the residue was taken in CHCl₃. The chloroform solution was washed with 5% NaHCO₃ solution, dried over anhydrous MgSO₄, filtered and evaporated the solvent to afford 3-hydroxylipoic hydrazide (0.82 g, 69%).

Synthesis of 3-hydroxylipoic-Methotrexate conjugate: Methotrexate (0.9 g, 2 mmol) and O-(7-Azabenzotriazol-1-yl)-N,N,N′,N′-tetramethyluronium hexafluoro phosphate, HATU (1.14 g, 3 mmol), N-methyl Morpholine (0.6 g, 6 mmol) were taken together in acetonitrile (25 mL) and DMF (2 mL) and into this solution 3-hydroxylipoic hydrazide (0.59 g, 2.5 mmol) was added and stirred for 16 hrs. The reaction was quenched with water and extracted with ethyl acetate (3×50 mL) and the organic layer was isolated, dried over anhydrous magnesium sulfate, filtered and evaporated to dryness. The crude product was purified through silica gel column chromatography to afford α-conjugate (0.19 g, 14%) and w-conjugate (0.76 g, 56%) in 1:4 ratio.

Synthesis of 3-O-acetyllipoic-Methotrexate conjugate: 3-hydroxylipoic-methotrexate conjugate (0.17 g, 0.25 mmol) was taken in acetic anhydride (1 mL) and into this solution was added catalytic amount of iodine. The reaction mixture was stirred further for 30 minutes and diluted with dichloromethane (50 mL) and ice crystal. The resulting solution was titrated with 0.1M sodium thiosulfate solution until the reaction mixture is colorless. The organic layer was separated, washed with water, dried over anhydrous magnesium sulfate, filtered and the solvent was evaporated under vacuum to afford the desired product as a white solid (0.14 g, 78%)

Synthesis of 3-fluorescein lipoic acid: 3-oxolipoic acid (0.22 g, 1 mmol) and fluorescein-5-thiosemicarbazide (0.42 g) were mixed together in acetonitrile (25 mL) and stirred for 16 hrs at room temperature. The reaction mixture forms homogeneous initially and the product precipitated out during the course of the reaction. The reaction mixture was filtered and dried under vacuum (0.35 g, 56%)

1. A compound of the Formula 1 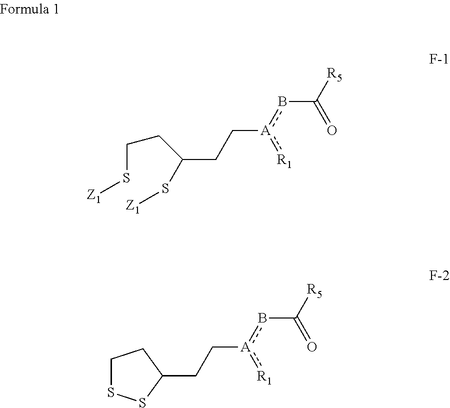
wherein: A and B are carbon atoms directly connected to carbon-carbon single bond, carbon-carbon double bond; Z₁ is H, COCH₃; R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙCₘH₂ₘ₊₁; —(CH₂CH₂CH₂O)ₙCₘH₂ₘ₊₁; —X(CH₂—CH₂O)ₙCₘH₂ₘ₊₁Y; X(CH₂CH₂CH₂O)ₙCₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₁; -alkene CₘH₂ₘ; -alkyne CₘH₂ₘ₋₂; —(CH₂CH₂O)ₙCₘH₂ₘY; —(CH₂CH₂CH₂O)ₙCₘH₂ₘY n is 0-16; m is 0-16; Y is H, OH, NH₂, SH, COOH; R₇ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R9 denotes R₆, R₇R₈, R₈; R₁₀ denotes R₆, R₇R₈, R₈;

2. The compound of claim 1 wherein: wherein: A and B are carbon atoms directly connected to carbon-carbon single bond, carbon-carbon double bond; Z₁ is H, COCH₃; R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙCₘH₂ₘ₊₁; —(CH₂CH₂CH₂O)ₙCₘH₂ₘ₊₁; —X(CH₂—CH₂O)ₙCₘH₂ₘ₊₁Y; X(CH₂CH₂CH₂O)ₙCₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₁; -alkene CₘH₂ₘ; -alkyne CₘH₂ₘ₋₂; —(CH₂CH₂O)ₙCₘH₂ₘY; —(CH₂CH₂CH₂O)ₙCₘH₂ₘY; n is 0-16; m is 0-16; Y is H, OH, NH₂, SH, COOH; R₇ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R9 denotes R₆, R₇R₈, R₈; R₁₀ denotes R₆, R₇R₈, R₈;

3. A method of reducing the symptoms associated with free radical mediated diseases comprising administering an effective amount of the compound of the Formula 1 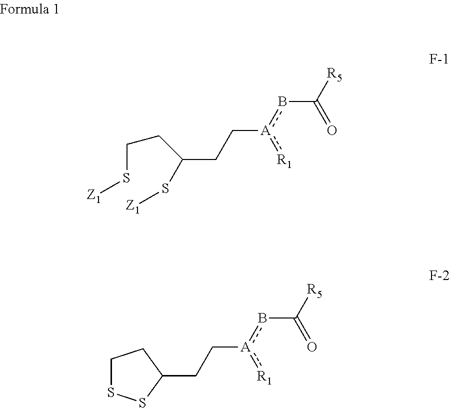
The compound of claim 1 wherein: A and B are carbon atoms directly connected to carbon-carbon single bond, carbon-carbon double bond; Z₁ is H, COCH₃; R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙCₘH₂ₘ₊₁; —(CH₂CH₂CH₂O)ₙCₘH₂ₘ₊₁; —X(CH₂—CH₂O)ₙCₘH₂ₘ₊₁Y; X(CH₂CH₂CH₂O)ₙCₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ (XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₁; -alkene CₘH₂ₘ; -alkyne CₘH₂ₘ₋₂; —(CH₂CH₂O)ₙCₘH₂ₘY; —(CH₂CH₂CH₂O)ₙCₘH₂ₘY; n is 0-16; m is 0-16; Y is H, OH, NH₂, SH, COOH; R₇ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R₉ denotes R₆, R₇R₈, R₈; R₁₀ denotes R₆, R₇R₈, R₈;

4. A method of treating free radical mediated diseases comprising administering an effective amount of the compound of the Formula 1 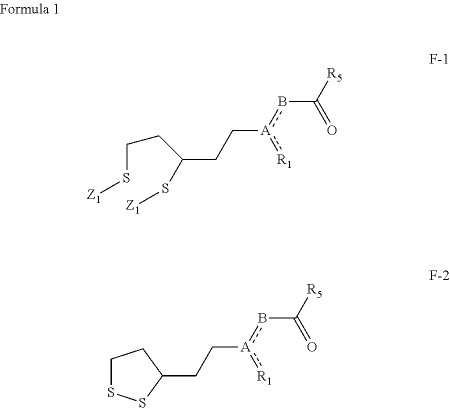
wherein: A and B are carbon atoms directly connected to carbon-carbon single bond, carbon-carbon double bond; Z₁ is H, COCH₃; R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙCₘH₂ₘ₊₁; —(CH₂CH₂CH₂O)ₙCₘH₂ₘ₊₁; —X(CH₂—CH₂O)ₙCₘH₂ₘ₊₁Y; X(CH₂CH₂CH₂O)ₙCₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ(XCH₂CH₂)ₙY; —(CH₂)ₘ(XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₁; -alkene CₘH₂ₘ; -alkyne CₘH₂ₘ₋₂; —(CH₂CH₂O)ₙCₘH₂ₘY; —(CH₂CH₂CH₂O)ₙCₘH₂ₘY; n is 0-16; m is 0-16; Y is H, OH, NH₂, SH, COOH; R₇ denotes (CH₂)ₘ(XCH₂CH₂)ₙY; —(CH₂)ₘ(XCH₂CH₂CH₂)ₙY; m=0-16, n=O-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol; R9 denotes R₆, R₇R₈, R₈; R₁₀ denotes R₆, R₇R₈, R₈;

5. The method of claims 2 to 4 wherein the free radical mediated disease is cancer, diabetic, polyneuropathy, liver cirrhosis or metal intoxication.

6. Fluorescent dye molecule containing Formula 1 compounds for the application towards a method of imaging in vitro. Tissue staining and detecting the free radical mediated diseases.

7. A method of magnesium source in the form of magnesium salt of lipoic acid having the Formula 1.

8. A method of vitamin source in the form of vitamin salt of lipoic acid having the formula 1.

9. A pharmaceutical composition comprising a composition of any claims 1 through 8 and a pharmaceutically acceptable carrier.

10. A method of inhibiting proliferation of diseased cells by contacting the diseased cells with an effective amount of Formula 1 compound that inhibits the diseased cells.
